(19) United States
(12) Patent Application Publication
(10) Pub. No.: US 2020/0082354 A1
(43) Pub. Date: Mar. 12, 2020

(54) SMART WASTE MANAGEMENT SYSTEM

(71) Applicant: HEMAL B. KURANI, Sunyvale, CA (US)

(72) Inventor: HEMAL B. KURANI, Sunyvale, CA (US)

(21) Appl. No.: 16/124,189

(22) Filed: Sep. 7, 2018

(51) Int. Cl. G06Q 10/00; G01F 23/00; G01C 21/00; G01K 1/02; G01J 1/42; G01N 33/00; G01P 13/00; G01W 1/02

(52) U.S. Cl. CPC G06Q 10/30; G01F 23/0007; G01C 21/005; G01W 1/02; G01J 1/4204; G01N 33/00; G01P 13/00; G01K 1/026

(57) ABSTRACT

A smart-waste management system includes a hardware and software system to accumulate and monitor waste data. The hardware includes sensor arrangements that gather information and wirelessly sends it to the software program. A smart-waste bin comprises of fill, location, temperature, humidity, odor, pollution, and ambient light sensors. In addition to this, the disclosed invention provides area map with live statistics and feedback from the monitored waste bins, such as information regarding the location, waste bin information, active litter lookout volunteers and performance. Using this information, garbage collection routes are optimized, and residents are provided with an incentive to reduce litter.

### FIELD OF THE INVENTION

This application relates generally to the field of waste management, and more specifically to a new system and method of the optimization of solid waste transportation and collection, as well as litter reduction.

### DESCRIPTION OF THE RELATED ART

Utter is most commonly defined as a piece of waste and rubbish that has been improperly disposed of; at the incorrect time, location, and without consent. Litter is very harmful and takes a negative toll on the environment. For example, it can pollute water and harm sea life, spread bacteria and disease, and start fires on hot days. Almost half of litter is unintentional, and it comes mostly from businesses, residents, and pedestrians. A few types of litter like plastic can stay in the environment for millions of years, ending up in bodies of water and forming debris. However, it can be collected effectively recycled or stored in landfills. Most people overproduce trash and litter, which damages and severely pollutes the environment.

The Smart-waste management system solves the problem of excessive littering by collecting and sending waste data through a wireless network, providing real time feedback to optimize garbage collection, thereby reducing litter.

The Smart-waste management system allows operators to efficiently identify, gather and transport solid and other waste to transfer or recycling facility centers for sorting, segregating, and recycling, while simultaneously preventing random dumping of waste. Smart-waste bin installed near the sites like schools, parks, strip malls, shopping malls, stadiums, and neighborhood helps eliminate littering by making the consumers potentially play enforcement roles themselves as litter lookout volunteers to produce desirable social behaviors in the community. They may also use the Trash picker or grabber to pick the litter and put it in the waste bin. Smart-waste bin includes fill, location, temperature, humidity, odor, pollution, motion, ambient light sensors and cameras to take picture of the trash in the smart-waste bin and pictures of surrounding litter.

### SUMMARY OF THE INVENTION

In one aspect, a smart-waste management system includes a waste bin storing a waste. The waste bin comprises a set of sensors attached on the waste bin. The set of sensors sending and receiving signals through a wireless network to a server system. The server system creating with the signals from the set of sensors a set of waste bin statistics for the waste bin. The server system uses the set of waste bin statistics to generate an optimal strategy for a waste removal service of the waste bin by one or several waste collection vehicles.

### BRIEF DESCRIPTION OF THE DRAWINGS

FIG. 1 illustrate an example overall smart-waste management system design to continuously monitor waste bins to optimize garbage collection and eliminate littering, according to some embodiments.

FIG. 2 depicts an exemplary smart waste management application workflow system that can be configured to perform any computations to optimize garbage collection provided herein.

FIG. 3 is a diagram of a sample smart-waste bin that can be utilized to implement various embodiments.

FIG. 4 is a diagram of an exemplary sample smart-waste bin microcomputer and sensors arrangement that can be utilized to implement various embodiments.

FIG. 5 illustrates an example set of smart waste management software architecture consisting of software user interface, methods, algorithms and database, according to some embodiments.

FIG. 6 illustrates an example software graphical user interface of the smart-waste management system with menu items, according to some embodiments.

FIG. 7 illustrates an example set of smart-waste management system software design components, according to some embodiments.

FIG. 8 illustrates an example block diagram of the smart waste management database design, according to some embodiments.

FIGS. 9 A-B illustrate an example process for operating a waste collection system in which a plurality of several waste bins receive waste; a server system that receives signals through a wireless network by a set of sensors on the plurality of waste bins to sense the status of one or several of the bins, according to some embodiments.

FIG. 10 depicts an exemplary computing system that can be configured to perform any one of the processes provided herein.

FIG. 11 is a block diagram of a sample computing environment that can be utilized to implement various embodiments.

The Figures described above are a representative set and are not an exhaustive with respect to embodying the invention.

### DESCRIPTION

The disclosed invention is a means of creating an optimized collection system of waste and reducing litter. In one embodiment, the smart-waste management system is two folded with a hardware and software system. The hardware includes a waste bin with smart microcomputer and sensors. The smart-waste bin sends data through a wireless network, to the smart waste management software. The software program includes different interactive user interfaces such as, inter alia: dashboard, waste bin street map and hot spots, waste bin details, residents, commercial, pedestrian, waste management facility etc. and that provide the detail information about each item.

The disclosed invention runs on an end to end application workflow. First, the real-time data of the waste bin, for example fill volume, location, temperature, humidity, odor, pollution, motion and ambient light values is collected. Simultaneously, the camera takes picture from inside and waste bin surroundings. The pedestrians and litter lookout volunteer can also upload the litter hot spot pictures to smart waste system. This litter picture information is also used to monitor the effectiveness of the residents and pedestrians in disposing the waste. Lastly, the smart-waste management system results are received through the software system.

In one aspect, there is a smart-waste management system consisting of many waste bins for accumulating waste. Sensors that are connected to the bin use a wireless network to send and receive data which is used to calculate effective routing strategies.

The Smart-waste management system additionally allows for residents to monitor their trash production and collectors to improve waste quantities, collection routes, and effectively use waste collection vehicles. This is achieved by disposing trash only from the containers that have reached their maximum threshold level, hence minimizing extra effort and resources.

Furthermore, the Smart-waste management system can reduce money spent for waste collection, decreasing the impact of waste on the environment.

The system can also improve public safety and hygiene practices when collection operators are notified to pick up the waste. This will prevent waste overflow, rotting, toxicity and foul air.

In addition to this, the disclosed invention can provide live statistics and feedback from the monitored waste bins, such as information regarding the waste bin location, waste fill volume, type of waste created, and foul smell usually from methane gas etc.

The disclosed invention enables residents to play enforcement roles by actively monitoring the litter in his or her community and potentially act as litter lookout volunteers to produce desirable social behaviors and reduce litter.

The waste collection system is also capable of sending air quality, temperature and humidity data to monitor air pollution, foul smell and toxicity in the surrounding environment and record data regarding the biodegraded and fermented state of the waste.

The waste collection system sensor arrangements can determine and compute the volume of waste in each bin, sending a signal to the server, notifying operators that the waste must be picked up.

The system sends the real-time vehicle and operator status information to the server and is stored in the database. This information is available to the user via the software application.

The schematic flow chart diagrams included herein are generally set forth as logical flow chart diagrams. As such, the depicted order and labeled steps are indicative of one embodiment.

### Exemplary Definitions

Application programming interface (API) can specify how software components of various systems interact with each other. The API can also be used to create mobile application to interact with smart waste management system.

BLUETOOTH is a wireless technology standard for exchanging data over short distances (e.g. using short-wavelength UHF radio waves in the ISM band from 2.4 to 2.485 GHz from fixed and mobile devices, and building personal area networks (PANs), etc. It is noted that other communication systems that transmit signals with messages from user's device to recipients can be used as well.

Cloud computing can involve deploying groups of remote servers and/or software networks that allow centralized data storage and online access to computer services or resources. These groups of remote serves and/or software networks can be a collection of remote computing services.

Camera is a device for recording visual images in the form of photographs, film, or video signals.

Commercial Customers is a segment of the business that is made up of commercial and industrial collection.

Landfill is a modern engineered way to deposit waste into the ground and still protect the environment.

Utter is small pieces of rubbish that have been left lying on the ground in public places. This includes trash, such as paper, cans, and plastic bottles, that is left lying in an open or public place.

Litter Lookout Volunteer is a person who monitors the litter in the neighborhood and identifies and uploads the litter pictures to the smart waste management system.

Methane is gas byproduct generated through natural decomposition of solid waste in landfills.

Route is a specifically directed course that a driver follows that has been designed for efficiency and to provide optimal service to customers.

Residential Customers is a segment of the collection business that is made up of single and multi-family dwellings.

Solid waste is a garbage from non-industrial sources, such as residential homes, restaurants, retail centers, and office buildings. It can include paper, discarded food items, plastics and other general discarded items.

Single board microcomputer is a complete computer built on a single board with microprocessors(s), memory, input/output (I/O), sensors and other features required of a functional computer.

Sensor is a device that detects or measures a physical property and records, indicates, or otherwise responds to it.

Transfer station is a facility that includes a large pad where residential and commercial collection vehicles empty the contents of their trucks. Other machinery (e.g. bulldozers) is then used to push the garbage into long-haul trailers for transport to disposal facilities.

Trash Picker or Grabber is a tool consisting of an arm to increase the range of person's reach when grabbing objects.

Waste Bin is a bin that holds rubbish until it is collected.

Waste collection vehicle or Garbage truck refers to a truck specially designed to collect solid and other waste and haul the collected waste to a solid waste treatment facility for recycling or landfill such as a landfill.

Waste collection operator is a person who drives sanitation vehicles and perform solid waste collection and disposal work for residential or commercial routes.

Waste picker is a person who picks reusable and recyclable materials thrown away by others to sell or for personal consumption.

### Exemplary Systems and Methods

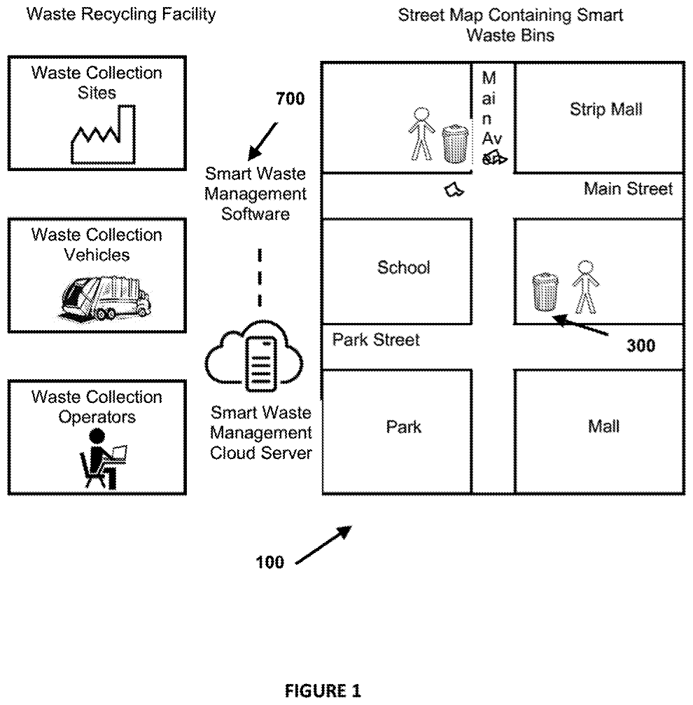

FIGS. 1-8 illustrate an example smart-waste management system 100, according to some embodiments. It includes information about various components such as waste collection sites, vehicles, operators, waste bin 300 and sensor hardware 400, residents, pedestrians, software, methods, algorithms, database and cloud computing server.

FIG. 1 depicts a street map 100 consisting of various components, according to some embodiments. Street map 100 is also the landing page view of the smart-waste management system mobile application and desktop application.

Referring to FIG. 1, there is shown an illustration of a garbage collection system street map, designed to optimize garbage collection and eliminate littering. Waste bin 300 sends and receives information collected by the one or several sensor arrangements, regarding waste bin location, waste fill volume, type of waste created, and/or foul smell. The data is processed through the smart waste management software 700, and sent to waste collection sites, waste collection vehicles, and operators to collect the waste received in each smart-waste bin.

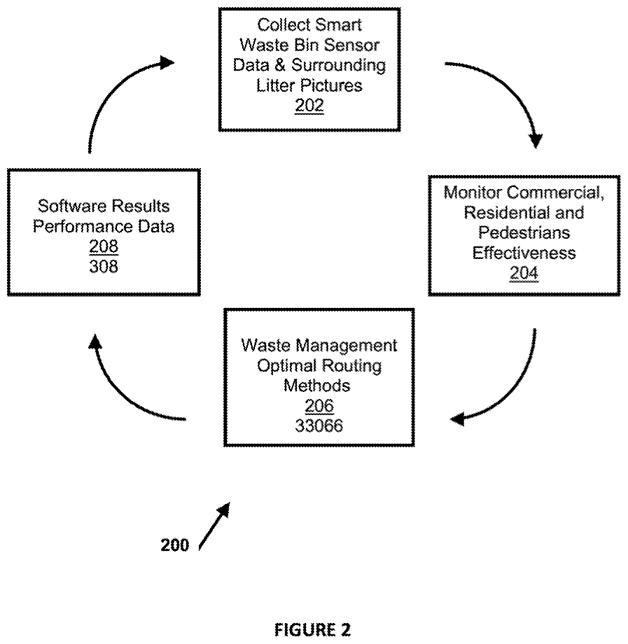

FIG. 2 depicts a smart waste management application workflow 200 of the process, according to some embodiments. In step 202, process 200 can collect smart-waste bin sensor data and surrounding litter pictures. In step 204, process 200 can monitor commercial, residential and pedestrian's effectiveness. In step 206, process 200 can implement waste management optimal routing methods. In step 208, process 200 can provide software results and analyze/display performance data.

In one example, the smart waste bins sensors data installed in any residential or commercial areas is continuously collected, monitored and sent to the cloud server. In addition, inside pictures of the waste bin as well as surrounding litter pictures are sent at regular intervals to the cloud server. Motion sensors can be programmed to send the data only when the trash or litter is thrown conserving the battery power of the device. This is followed by processing the sensor data by the cloud server and optimizing the waste collection vehicles routing to pick up the waste at optimal cost. All this results are available through smart-waste management system software 700 on a desktop or mobile devices.

Various optimization methodologies can be implemented herein. For example, machine-learning techniques can be used for optimization processes. Machine learning is a type of artificial intelligence (Ai) that provides computers with the ability to learn without being explicitly programmed. Machine learning focuses on the development of computer programs that can teach themselves to grow and change when exposed to new data. Example machine learning techniques that can be used herein include, inter alia: decision tree learning, association rule learning, artificial neural networks, inductive logic programming, support vector machines, clustering, Bayesian networks, reinforcement learning, representation learning, similarity and metric learning, and/or sparse dictionary learning. Historical user data, set of other user data that are similar to the user, all historical user data, etc. can be used as training data sets.

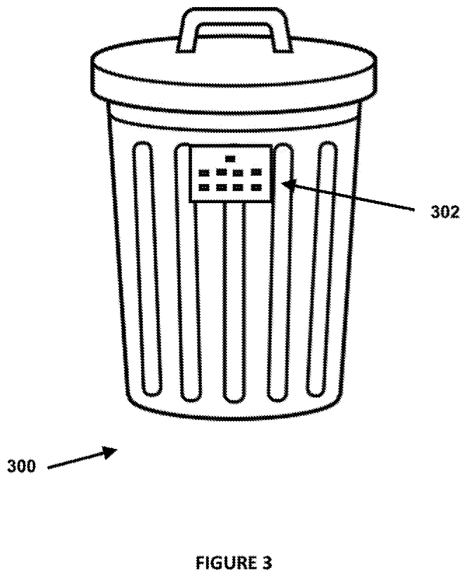

FIG. 3 is a diagram of a sample smart-waste bin 300 that can be utilized to implement various embodiments and receive waste. Smart-waste bin 300 includes sensors 302. Sensors 302 can send and receive data via various computer networks (e.g. Wi-Fi, the Internet, cellular networks, etc.). Sensors 302 arrangements are attached on top or to the perimeter of smart-waste bin 300 (and/or any other location which would not block the pickup and disposal of the waste).

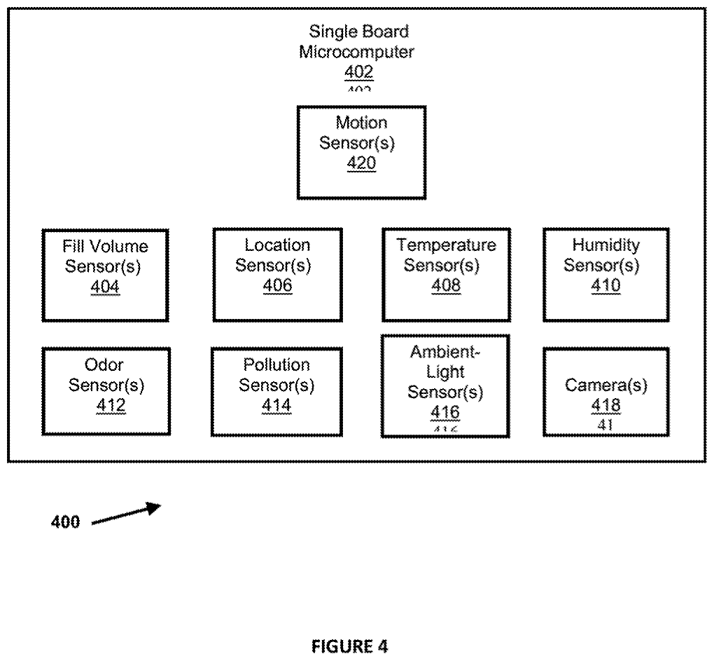

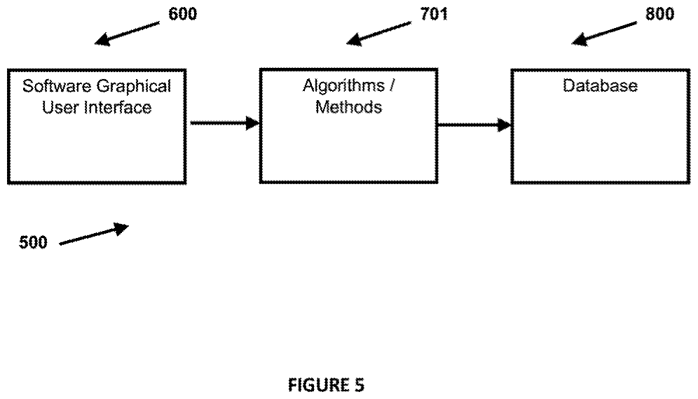

FIG. 4 is a diagram of an example smart-waste bin microcomputer and sensor arrangement 400 with various embodiments. Smart-waste bin microcomputer and sensor arrangement 400 can be utilized for providing live statistics and feedback. Smart-waste bin microcomputer and sensor arrangement 400 can include a small single board microcomputer 402 wherein the various the sensors are built. The sensors can include, inter alia: one or more fill-volume sensors 404 to calculate the volume of waste received; one or more location sensors 406 to provide GPS coordinates; one or more temperature sensors 408, one or more humidity sensors 410, one or more odor sensors 412, one or more pollution sensors 414 to check for bio degrading and fermenting waste, one or more smart cameras 416 to obtain digital images/videos of the surrounding litter, one or more ambient light sensors 418 to conserve the battery during night and/or other period of inactive use of smart-waste bin (e.g. smart-waste bin microcomputer and sensor arrangement 400 can be set in a low-energy sleep mode during the night using ambient light sensor(s) 416, motion sensor(s) 420 etc.).

FIG. 5 illustrates an example of smart waste management software architecture 500, according to some embodiments. Smart waste management software architecture 500 can process the collected sensor data stored in the database 800. Smart waste management software architecture 500 can present the collected sensor data to the user by various methods and algorithms 701. The software user interface can display the performance statistics. Algorithms 701 can analyze and process the collected sensor data. Collected sensor data is stored in a smart waste management database (e.g. database 800). The performance statistics can be accessed by client applications and/or other user-side computing system via internet, intranet, WIFI and/or Bluetooth.

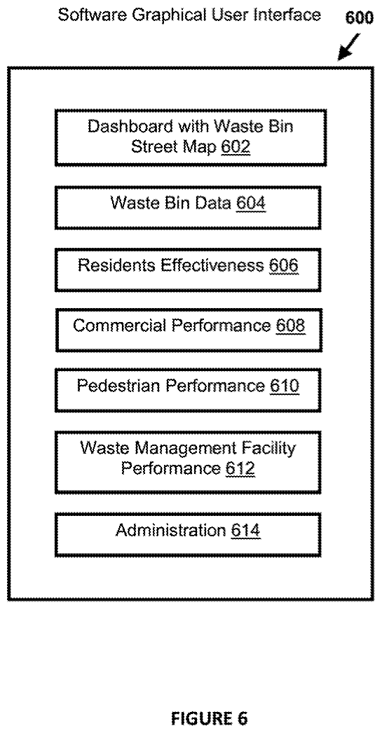

FIG. 6 illustrates an example software graphical user interface 600 which is user activated. It includes various menu items such as the following. Dashboard with waste bin street map 602 screen displays a street map of the commercial and residential user's personal waste bin in the form of an icon. When the user hovers over the icon, a complete set of details is provided. Waste bin street map 602 also includes litter hotspots and waste bin fill status and all the other sensor data. The waste management company's dashboard includes a map of the area, routes, current location of vehicles, litter hotspots, residential and commercial waste bin volume information when the icon is hovered over the location. The software includes an application programmer interface (API) to allow a user to create their own applications.

Waste bin data screen 604 provides information regarding waste bin fill volume, location coordinates, temperature, humidity, odor, and pollution data. Waste bin data screen 604 also displays the time lapsed pictures of inside trash and outside litter pictures.

Residents effectiveness screen 606 includes specifically of residential customer profiles, wherein the address and the waste bin volume and other sensors data is displayed. Residents effectiveness screen 606 also displays the waste records data in weekly, monthly, and yearly in a graphical and tabular form.

Commercial performance 608 screen includes commercial customer profiles. Each commercial customer profile can include the address and the waste bin volume and other sensors data is displayed. Commercial performance 608 also displays the waste records data in weekly, monthly, and yearly in a graphical and tabular form.

Pedestrian performance screen 610 provides the complete profile of active pedestrians and litter lookout volunteers. Pedestrian performance screen 610 provides interface to take litter pictures, categorize and map them, and either dispose the litter to the nearest waste bin or alert the waste management company. According to the number of pictures taken, uploaded and disposed, the pedestrian may gain recognition reward points.

Waste management facility performance screen 612 provides information of the waste or transfer station facility location, number of vehicles and operators. The total cost method includes variables such as the number of commercial and residential waste bins, total volume in each waste bin, the number of operators and vehicles, and route length. This data allows waste management companies to optimize the number of vehicles, operators, and pickup routes. Waste management facility performance screen 612 also displays in the graphical tabular form the aggregate waste bin volumes in weekly, monthly, and yearly waste.

Administration screen 614 includes the user's ability to create, modify, and delete the current user profile. Administration screen 614 also has a search capability and provides lists of the administrators and other users.

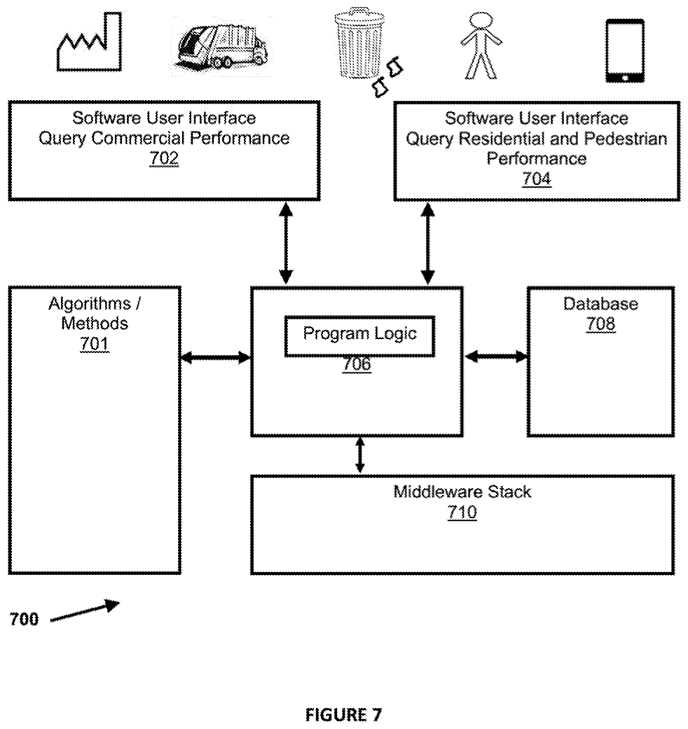

FIG. 7 illustrates an example set of smart-waste management system software design 700, according to some embodiments. The smart waste bins send the sensor data to the web server. The data is processed through specific algorithms and methods 701 in this web server and sent to a database 708 to be stored. The data can be accessed by the user thorough the software user interfaces (e.g. software user interface query commercial performance 702 and 704). Middleware stack 710 allows methods to provide outputs and communicate rapidly, finally displaying the data on the software application. Web server 706 can include program logic.

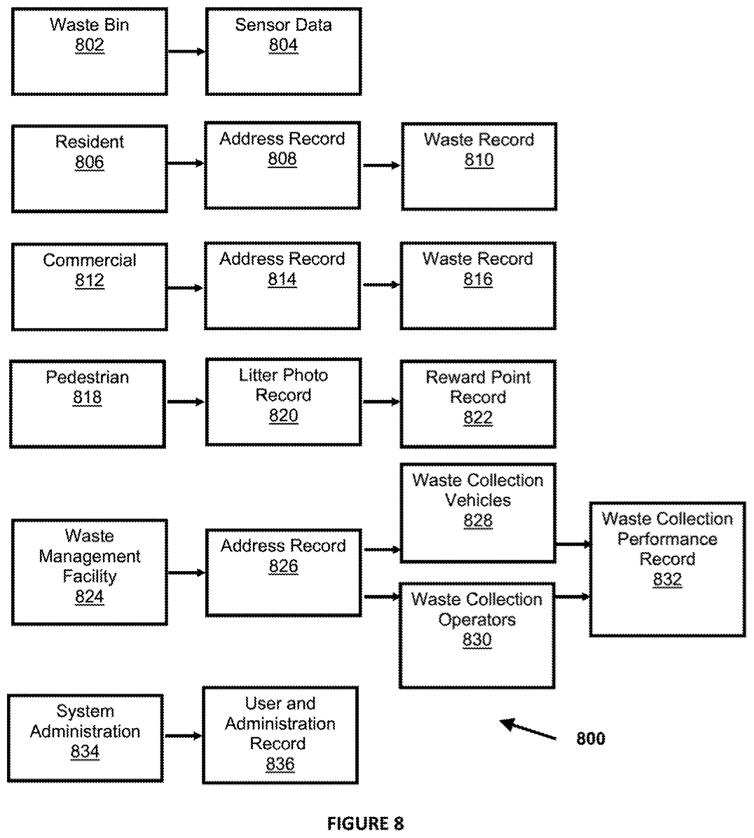

FIG. 8 illustrates an example block diagram of the smart waste management database design 800, according to some embodiments. Smart waste management database design 800 can correspond to the software architecture in FIG. 6. The waste bin data table 802 stores data regarding the waste bin and sensor information 804. The resident data table 806 stores complete resident profile, address record 808 and resident waste record 810. The commercial data table 812 stores complete commercial profile, address record 814 and commercial waste record 816. The pedestrian performance data table 818 stores a record of litter digital photos 820 and reward point recognitions 822. The waste management facility performance data table 824 stores complete facility profile, address record 826, waste collection vehicles 828, waste collection operators 830, waste pickers, and overall waste collection performance record 832. Finally, the system administration screen data table 834 stores information regarding the user and administration login and password and audit trails records 836.

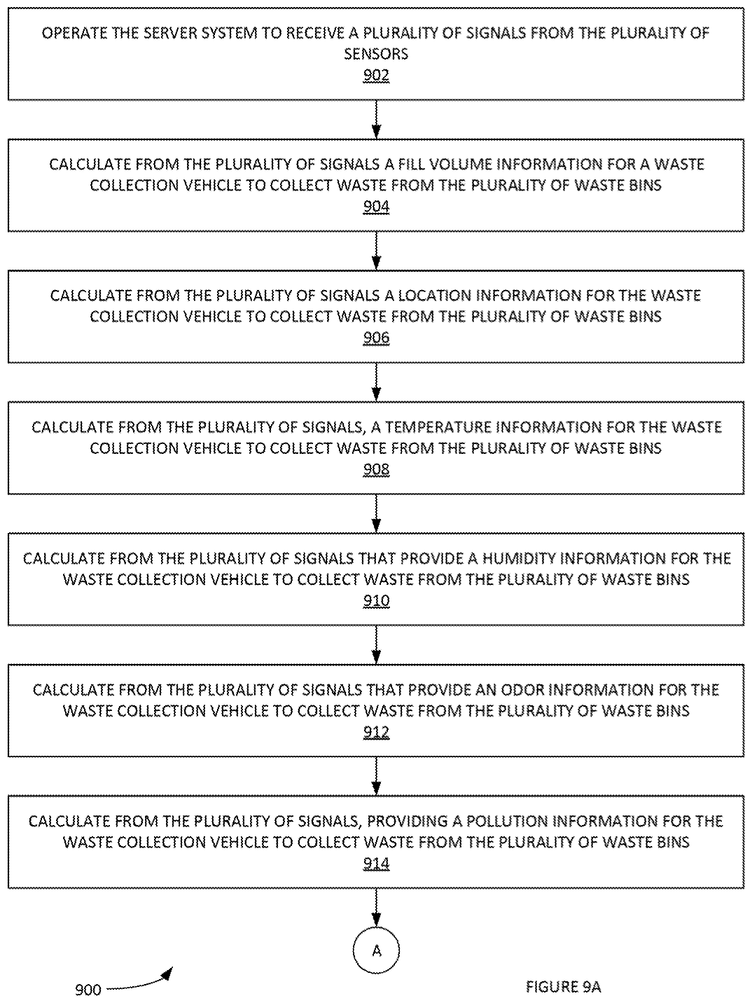

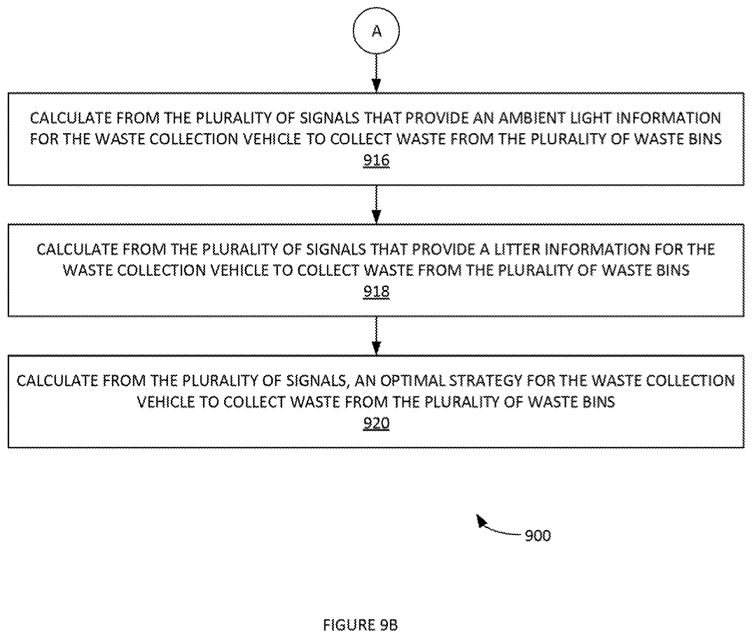

FIGS. 9 A-B illustrate an example process 900 for operating a waste collection system in which a plurality of several waste bins receive waste; a server system that receives signals through a wireless network by a set of sensors on the plurality of waste bins to sense the status of one or several of the bins, according to some embodiments. In step 902, process 900 can operate the server system to receive a plurality of signals from the plurality of sensors. In step 904, process 900 can calculate from the plurality of signals a fill volume information for a waste collection vehicle to collect waste from the plurality of waste bins. In step 906, process 900 can calculate from the plurality of signals a location information for the waste collection vehicle to collect waste from the plurality of waste bins. In step 908, process 900 can calculate from the plurality of signals, a temperature information for the waste collection vehicle to collect waste from the plurality of waste bins. In step 910, process 900 can calculate from the plurality of signals that provide a humidity information for the waste collection vehicle to collect waste from the plurality of waste bins. In step 912, process 900 can calculate from the plurality of signals that provide an odor information for the waste collection vehicle to collect waste from the plurality of waste bins. In step 914, process 900 can calculate from the plurality of signals, providing a pollution information for the waste collection vehicle to collect waste from the plurality of waste bins. In step 916, process 900 can calculate from the plurality of signals that provide an ambient light information for the waste collection vehicle to collect waste from the plurality of waste bins. In step 918, process 900 can calculate from the plurality of signals that provide a litter information for the waste collection vehicle to collect waste from the plurality of waste bins. In step 920, process 900 can calculate from the plurality of signals, an optimal strategy for the waste collection vehicle to collect waste from the plurality of waste bins.

### Additional Example Methods

Ultrasonic sensor includes an ultrasonic transmitter, receiver and control circuit. The transmitter transmits short bursts of signals which gets reflected by target and are picked up by the receiver. The time difference between transmission and reception of the ultrasonic signals is calculated. First using the speed of sound and ‘Speed=Distance/Time’ equation, the distance between the source and target is calculated. After this a specialized algorithm calculates the fill volume based on distance and waste bin dimensions. returning the fill volume information in percent and sending the data to the software graphical user interface. The method uses the waste bin parameters such as length, inner diameter, outer diameter, camera pictures. When the waste bin threshold fill volume is reached, an alert is sent to collection vehicles to collect waste.

A Global Positioning System (GPS) receiver in the waste bin calculates its position by precisely timing the signals sent by GPS satellites in orbit. The location sensor includes input GPS information. The waste bin receiver uses the messages it receives to determine the transit time of each message. It then computes the distance to each satellite. The distances along with the satellites precise locations are used to calculate the location of the waste bin receiver unit using a process called trilateration. A method is used to ensure there is no interference due to trash or litter around the waste bin or location of the waste bin especially when it is located indoors. The data is transmitted to the software graphical user interface to be displayed.

The temperature and humidity sensor are made of two parts, temperature sensing device whose resistance changes with temperature. Humidity sensors work by detecting changes that alter electrical currents or temperature in the air. A method consisting algorithm based on the temperature, humidity, litter picture, amount of garbage is used to calculate the rotting risk index. An alert is sent to one or several waste collection vehicles to collect waste if signs of methane and rotting are present.

The odor sensor includes poisonous gas inputs through an arrangement of gas sensor arrays. The sensor surface includes materials that could respond to the odorous compounds differently. These sensors are used as receptors like electronic nose which receive various molecules to associate smells. A method for a chemical detection mechanism in which odor risk index information is calculated through an algorithm. It sends the odor data over wireless network to the server. An alert is sent to one or several waste collection vehicles if signs of air toxicity is present.

The pollution sensor includes input infrared radiation signals, and an infrared light source. The wave lengths of the signals received by the infrared sensor are evaluated. Based on this, the concentration of carbon dioxide is calculated through an algorithm. A method is used to calculate the pollution risk index based on parameters like temperature, humidity, and signal drift. A return value is sent to the database and is displayed on the software graphical user interface.

The ambient light sensor is used to conserver the battery life of the smart waste management system. It detects the brightness of light it receives and converts it to a numerical value. The charges of the electrons and wave lengths causes the light energy to be converted into digital electrical signals. An algorithm calculates the corresponding change in voltage for the detection of light. Digital camera and litter pictures are not taken if it is completely dark and goes into sleep mode. The ambient light sensor data is sent to the server.

The smart camera includes input microscopic light detectors that capture light though an image detecting sensor array. An algorithm converts the picture into a digital format and immediately transmits the image to an internet router to be displayed on the software graphical user interface. A feature extraction method is used to differentiate between different type of trash in the waste bin. The optimized feature extracted file is also transmitted to the server. The system also provides litter information and images around the waste bin for collection vehicles to pick up the waste.

The smart waste management facility performance mathematical model includes an algorithm to calculate the cost function per square mile. The cost function calculation is based on the number of commercial waste bins multiplied by the cost to pick up the commercial waste; added to the number of residential waste bins, multiplied by the cost to pick up the residential waste; added to the volume of garbage in the waste bins, multiplied by the cost it takes to recycle and dispose of the waste. It also considers several other factors like number of collection waste facilities, operators, location of the facility and other operational cost. The waste performance data allows for selection of the best optimal cost strategy to collect and dispose of the waste.

### Example Computer Architecture and Systems

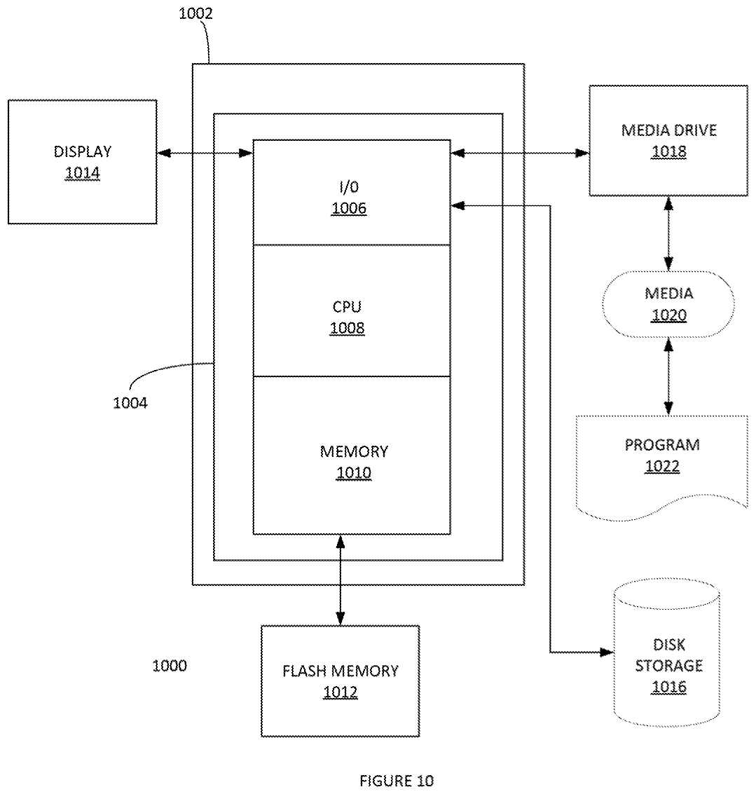

FIG. 10 depicts an exemplary computing system 1000 that can be configured to perform any one of the processes provided herein. In this context, computing system 1000 may include, for example, a processor, memory, storage, and I/O devices (e.g., monitor, keyboard, disk drive, Internet connection, etc.). However, computing system 1000 may include circuitry or other specialized hardware for carrying out some or all aspects of the processes. In some operational settings, computing system 1000 may be configured as a system that includes one or more units, each of which is configured to carry out some aspects of the processes either in software, hardware, or some combination thereof.

FIG. 10 depicts computing system 1000 with a number of components that may be used to perform any of the processes described herein. The main system 1002 includes a motherboard 1004 having an I/O section 1006, one or more central processing units (CPU) 1008, and a memory section 1010, which may have a flash memory card 1012 related to it. The I/O section 1006 can be connected to a display 1014, a keyboard and/or other user input (not shown), a disk storage unit 1016, and a media drive unit 1018. The media drive unit 1018 can read/write a computer-readable medium 1020, which can contain programs 1022 and/or data. Computing system 1000 can include a web browser. Moreover, it is noted that computing system 1000 can be configured to include additional systems in order to fulfill various functionalities. Computing system 1000 can communicate with other computing devices based on various computer communication protocols such a Wi-Fi, Bluetooth® (and/or other standards for exchanging data over short distances includes those using short-wavelength radio transmissions), USB, Ethernet, cellular, an ultrasonic local area communication protocol, etc.

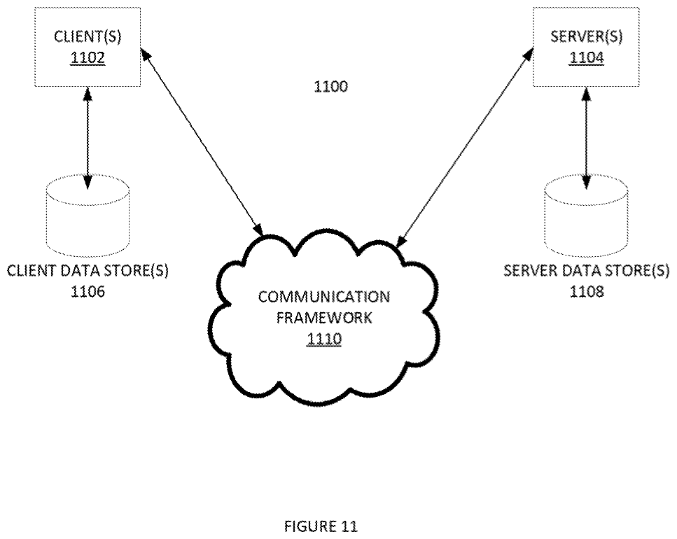

FIG. 11 is a block diagram of a sample computing environment 1100 that can be utilized to implement various embodiments. The system 1100 further illustrates a system that includes one or more client(s) 1102. The client(s) 1102 can be hardware and/or software (e.g., threads, processes, computing devices). The system 1100 also includes one or more server(s) 1104. The server(s) 1104 can also be hardware and/or software (e.g., threads, processes, computing devices). One possible communication between a client 1102 and a server 1104 may be in the form of a data packet adapted to be transmitted between two or more computer processes. The system 1100 includes a communication framework 1110 that can be employed to facilitate communications between the client(s) 1102 and the server(s) 1104. The client(s) 1102 are connected to one or more client data store(s) 1106 that can be employed to store information local to the client(s) 1102. Similarly, the server(s) 1104 are connected to one or more server data store(s) 1108 that can be employed to store information local to the server(s) 1104. In some embodiments, system 1100 can instead be a collection of remote computing services constituting a cloud-computing platform.

### CONCLUSION

Although the present embodiments have been described in reference to specific example embodiments, different modifications can be made to these without changing or taking away from the broader objective of the design. For example, additional sensors, devices, modules, or alterations in the software can be operated to improve the system.

In addition, it can be appreciated that the various operations, processes, and methods disclosed herein can be embodied in a machine readable medium and/or a machine accessible medium compatible with a data processing system and can be performed in any order. Accordingly, the specification and drawings are to be regarded in an illustrative rather than a restrictive sense. In some embodiments, the machine-readable medium can be a non-transitory form of machine-readable medium.

What is claimed as new and desired to be protected by Letters Patent of the United States is:

1. A smart-waste management system comprising:
a waste bin storing a waste, wherein the waste bin comprises a set of sensors attached on the waste bin;
the set of sensors sending and receiving signals through a wireless network to a server system; and
the server system creating with the signals from the set of sensors a set of waste bin statistics for the waste bin, wherein the server system uses the set of waste bin statistics to generate an optimal strategy for a waste removal service of the waste bin by one or several waste collection vehicles.

2. The smart-waste management system of claim 1, wherein set of sensors attached on the waste bin comprises a waste-bin fill volume sensor to determine a waste-bin fill volume, a waste-bin location sensor to determine a waste-bin location, a waste-bin temperature sensor to determine a waste-bin temperature, a waste-bin humidity sensor to determine a waste-bin humidity value, a waste-bin odor sensor to determine a waste-bin odor, a waste-bin pollution sensor to determine a waste-bin pollution, a waste-bin ambient light sensor to determine a waste-bin ambient light, a waste-bin motion detector and a waste-bin digital camera, and wherein the set of sensors sends data to the server system.

3. The smart-waste management system of claim 2, wherein the waste-bin fill volume sensor sends a volume data of the waste bin to the server system.

4. The smart-waste management system of claim 3, wherein the waste-bin location sensor sends a geo-spatial position of the waste bin to the server system.

5. The smart-waste management system of claim 4, wherein the waste-bin temperature sensor sends a temperature surrounding the waste bin to the server system.

6. The smart-waste management system of claim 5, wherein the waste-bin humidity sensor sends a humidity value surrounding the waste bin to the server system.

7. The smart-waste management system of claim 6, wherein the waste-bin odor sensor sends a toxicity data around the waste bin to the server system.

8. The smart-waste management system of claim 7, wherein the waste-bin pollution sensor sends an air quality data of the waste bin to the server system.

9. The smart-waste management system of claim 8, wherein the waste-bin ambient light sensor sends an ambient light level proximate to the waste bin or an illuminance value of the waste bin to the server system.

10. The smart-waste management system of claim 9, wherein the waste-bin camera sends digital images of a litter around the waste bin and a digital image of the waste inside the waste bin to the server system.

11. The smart-waste management system of claim 10, wherein software system comprises a user interface, a waste-pickup means and a database that provides a waste-bin data to a user via the user interface.

12. The smart-waste management system of claim 11, wherein the waste-bin motion detector determines that a user is in a specified distance of the waste bin for a specified period of time, and wherein the waste-bin motion detector places the plurality of sensors in an energy-saving sleep state.

13. The smart-waste management system of claim 12, wherein while the plurality of sensors are in the energy-saving sleep state the waste-bin odor sensor and the waste-bin humidity sensor continue to operation in an active state.

14. The smart-waste management system of claim 13, wherein the waste-bin motion detector determines that a user is in a specified distance of the waste bin, and wherein the waste-bin motion detector places the plurality of sensors in an active state.

15. The smart-waste management system of claim 14, the server system obtains a pollution report from a weather database to augment output of the waste-bin pollution sensor.

16. A method of operating a waste collection system in which a plurality of several waste bins receive waste; a server system that receives signals through a wireless network by a set of sensors on the plurality of waste bins to sense the status of one or several of the bins, wherein the method includes:
operating the server system to receive a plurality of signals from the plurality of sensors;
calculating from the plurality of signals a fill volume information for a waste collection vehicle to collect waste from the plurality of waste bins;
calculating from the plurality of signals a location information for the waste collection vehicle to collect waste from the plurality of waste bins;
calculating from the plurality of signals, a temperature information for the waste collection vehicle to collect waste from the plurality of waste bins;
calculating from the plurality of signals, providing a humidity information for the waste collection vehicle to collect waste from the plurality of waste bins;
calculating from the plurality of signals, providing an odor information for the waste collection vehicle to collect waste from the plurality of waste bins;
calculating from the plurality of signals, providing a pollution information for the waste collection vehicle to collect waste from the plurality of waste bins;
calculating from the plurality of signals, providing an ambient light information for the waste collection vehicle to collect waste from the plurality of waste bins;
calculating from the plurality of signals, providing a litter information for the waste collection vehicle to collect waste from the plurality of waste bins; and
calculating from the plurality of signals, an optimal strategy for the waste collection vehicle to collect waste from the plurality of waste bins.

17. A method as claimed in claim 16 further comprising:
providing the waste collection vehicle with a set of waste bins statistics for each waste bin of the plurality of waste bins to modify the current optimal strategy in real-time during a collection of the waste from the plurality of waste bins.

18. A method as claimed in claim 17 further comprising:
providing the waste bin location information to a pedestrian to correctly dispose waste in the waster bin.

19. A method as claimed in claim 18 further comprising:
providing the plurality of waste bin information to the pedestrian and a litter lookout volunteer to produce a desirable social behaviors in a community.
